(19) United States
(12) Patent Application Publication
(10) Pub. No.: US 2006/0205795 A1
(43) Pub. Date: Sep. 14, 2006

(54) Cancer treatment with epothilones

(76) Inventors: David Parkinson, Westlake Village, CA (US); ivo Sklenar, Oberwil (CH); Sara Zaknoen, Hoboken, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/561,757

(22) PCT Filed: Jun. 25, 2004
(86) PCT No.: PCT/EP04/06921

§ 371 Date: Apr. 5, 2006

Related U.S. Application Data
(60) Provisional application No. 60/483,263, filed on Jun. 27, 2003.

(51) Int. Cl. A61K 31/427

(52) U.S. Cl. 514/365

(57) ABSTRACT

The use of an epothilone for the treatment of a proliferative disease comprising the step of daily administration of a therapeutically effective amount of an epothilone by continuous intravenous (i.v.) administration.

### SUMMARY OF THE INVENTION

The invention relates to the treatment of a proliferative disease, especially according to certain treatment regimens using an epothilone, especially epothilone B; preferably of a gastrointestinal tumor, more preferably (1) a tumor of the colon and/or the rectum (colorectal tumor), especially if it is refractory to 5-FU and at least one standard treatment with an other chemotherapeutic, especially irinotecan or oxaliplatin; (2) a tumor of the genitourinary tract, preferably a tumor of the prostate, including primary and metastatic tumors, especially If refractory to hormone treatment (“hormone refractory prostate cancer”) and/or treatment with other standard chemotherapeutics; more preferably a tumor of the ovary, including primary and metastatic tumors, especially if refractory to platinum (“platinum refractory ovarian cancer”) and/or taxane treatment and/or treatment with other standard chemotherapeubcs; (3) an epidermoid tumor, more preferably an epidermoid head and neck tumor, most preferably a mouth tumor; (4) a lung tumor, more preferably a non-small cell lung tumor, especially any of these tumors that is refractory to treatment with one or more other chemotherapeutics (especially due to multidrug resistance), especially to treatment with a member of the taxane class of anti-cancer agents, in particular TAXOL®; or (5) a breast tumor, more preferably one that is multidrug resistant, especially refractory to treatment with a member of the taxane class of anti-cancer agents, in particular TAXOL®; relating especially also to the treatment of a multidrug resistant lung tumor (preferably a non-small cell lung tumor), a multidrug resistant breast tumor, or a multidrug resistant epidermoid tumor, or in a broader sense of the invention to a treatment schedule for the treatment of an aforementioned or (in a broader sense of the invention) any other tumor, especially if it is refractory to one or more chemotherapeutics, especially multidrug resistant and/or TAXOL® refractory), such as a melanoma, ovarian cancer, pancreas cancer, neuroblastoma, head and neck cancer or bladder cancer, or in a broader sense renal, brain or gastric cancer; by administration of an epothilone as a cytotoxic agent, especially epothilone B; the term “treatment” also encompassing (i) a method of treatment for (=for treating of) said disease comprising administration of said cytotoxic agent (preferably an epothilone, especially epothilone B, in each case preferably together with a pharmaceutically acceptable carrier) to a warm-blooded animal, especially if in need of such treatment, in a therapeutically effective amount, in at least one treatment; (ii) the use of said cytotoxic agent, for the treatment of a proliferative disease; (iii) the use of said cytotoxic agent for the manufacture of a pharmaceutical preparation for the treatment of said proliferative disease (comprising admixing said cytotoxic agent with a pharmaceutically acceptable carrier); (iv) a pharmaceutical preparation comprising a dose of said cytotoxic agent that is appropriate for the treatment of said proliferative disease. The invention is, in a preferred embodiment, directed to the treatment of (human) patients or patient groups where other treatments, especially standard treatment with an other chemotherapeutic, especially 5-fluorouracil; or therapy with members of the taxane class of anti-cancer agents, such as TAXOL®, has failed. It also relates to an epothilone, especially epothilone B, for use in the treatment of a proliferative disease, especially where said disease is refractory to treatment with a standard therapeutic.

### BACKGROUND OF THE INVENTION

Cancer still represents a major unmet medical need. Initial treatment of the disease is often surgery, radiation treatment or the combination, but recurrent (metastatic) disease is common. Chemotherapeutic treatments for most cancers are generally not curative, but only delay disease progression. Commonly, tumors and their metastases become refractory to chemotherapy, in an event known as development of multidrug resistance. In many cases, tumors are inherently resistant to some classes of chemotherapeutic agents [see DeVita V. T., Principles of Cancer Management: Chemotherapy. In: Cancer. Principles and Practice of Oncology. DeVita V. T. et al (eds.), 5th edition, Lippincott-Raven, Philadelphia, New York (1977), pp. 333-347; or Cleton, F. J., Chemotherapy: general aspects. In: Oxford Textbook of Oncology; Peckham, M., et al, Oxford University Press, Oxford, New York, Tokyo (1995), Vol. 1, pp. 445-453]. This is, for example, the case for lung tumors, especially non-small cell lung carcinoma, or also for epidermoid tumors, like epidermoid head and neck, especially mouth, tumors, or also for breast tumors. Other mechanisms why tumors are not treatable (are refractory to treatment) can be, for example, the presence of tubulin mutations or glutathione mediated mechanisms.

Intestinal, especially colorectal, cancer defines a special case of the unmet medical needs in cancer treatment. Initial treatment of the disease is often surgery, radiation treatment or the combination, but recurrent (metastatic) disease is common. First-line chemotherapeutic treatments for recurrent colorectal cancer include 5-fluorouracil, leucovorin, irinotecan and possibly oxaliplatin. But this treatment provides at best delay of disease progression as the tumors usually become refractory to treatment. Chemotherapy of this refractory stage of disease involves other classical cytotoxic agents, but are all considered inadequate [see Cohen et al., Cancer of the colon. In: Cancer. Principles and Practice of Oncology; DeVita et al. (eds.), 5th edition, Lippincott Raven. Philadelphia, New York 1997, pp. 1144-1197; or Rowinsky, Ann. Rev. Med. 48, 353-74 (1997)]Also for cancer of the genitourinary tract, especially ovarian and prostate cancers, a further unmet medical need, initial treatment is as mentioned above for colorectal cancer, showing similar problems. First-line chemotherapeutic treatment for recurrent prostate cancer includes anti-androgens, and the recurrence is frequently androgen-dependent. But this treatment provides only delay of disease progression as the tumors almost always become refractory to anti-androgens within 6 months to 2 years (hormone-refractory prostate tumors). Chemotherapy of this anti- androgen refractory stage of diseases involves mitoxantrone or other classical anticancer cytotoxic agents, but all are considered as inadequate [see Oesterling et al., Cancer of the prostate. In: Cancer. Principles and Practice of Oncology. DeVita, V. T., et al. (eds.), 5th edition, Lippincoft-Raven, Philadelphia, New York 1997, pp 1322-86; Sternberg, Cancers of the genitourinary tract. In: Cavalli et al. (eds.), Textbook of Medical Oncology; or Roth, B. J., Semin. Oncol. 23(6 Suppl. 14), 49-55 (1996)]. First line treatment of ovarian cancer includes surgery and chemotherapy with a combination including a taxane and a platinum. However, the majority of Stage III and Stage IV patients will recur and subsequent treatment is ineffective in prolonging life. DeVita, V. T., et al. (eds.), 5th edition, Lippincott-Raven, Philadelphia, New York 1997, pp 1322-86; Sternberg, Cancers of the genitourinary tract. In: Cavalli et al. (eds.), Textbook of Medical Oncology; or Roth, B. J., Semin. Oncol. 23(6 Suppl. 14), 49-55 (1996)].

Among cytotoxic agents for the treatment of tumors, TAXOL® (paclitaxel), a microtubule stabilizing agent, has become a very important compound with a remarkable economic success [see Rowinsky E. K., The development and clinical utility of the taxane class of antimicrotubule chemotherapy agents; Ann. Rev. Med. 48, 353-374 (1997)]. Taxotere® (docetaxel) is a further microtubule stabilizing agent which is also an important compound.

However, TAXOL® and Taxotere® have a number of disadvantages. Especially their extremely low solubility in water represents a severe problem. It has become necessary to administer TAXOL® in a formulation with Cremophor EL® (polyoxyethylated castor oil; BASF, Ludwigshafen, Germany) which has severe side effects, causing inter alia allergic reactions that in one case even were reported to have led to the death of a patient. More severely, certain tumor types are known to be refractory to treatment with TAXOL® and Taxotere® even when the drug is administered as front-line therapy, or the tumors develop resistance to TAXOL® or Taxotere after multiple cycles of exposure.

Although the taxane class of antimicrotubule anti-cancer agents has been hailed as the “perhaps most important addition to the chemotherapeutic armamentarium against cancer over the past several decades” [see Rowinsky E. K., Ann. Rev. Med. 48, 353-374 (1997)] and despite the commercial success of TAXOL®, there remain limitations to TAXOL®'s efficacy. TAXOL® treatment is associated with a number of significant side effects and some major classes of solid tumors, namely colon and prostate, are poorly responsive to this compound (see Rowinsky E.K., loc. cit.). Specifically, as a single agent, TAXOL® has been considered to be poorly active clinically in colorectal, renal, prostatic, pancreatic, gastric and brain cancers [see Rowinsky E.K., loc. cit.; Bitton, R. J., et al., Drug Saf. 12, 196-208 (1995); or Arbuck, S. G., et al., J. Natl. Cancer Inst. Monogr. 15, 11-24 (1993)]. For example, the effectiveness of TAXOL® can be severely limited by acquired drug resistance mechanisms occurring via various mechanisms, such as overexpression of phosphoglycoproteins that function as drug efflux pumps.

Therefore, there exists an urgent need to find compounds and appropriate dosing regimens with these compounds to expand the armamentarium of cancer treatment, especially in the majority of cases where treatment with taxanes and other anticancer compounds is not associated with long term survival.

The epothilones, especially epothilones A and B, represent a new class of microtubule stabilizing cytotoxic agents (see Gerth, K. et al., J. Antibiot. 49, 560-3 (1996); or Hoefle et al., DE 41 38 042), e.g. with the formulae: 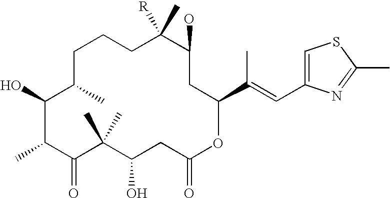
wherein R is hydrogen (epothilone A) or methyl (epothilone B).

These compounds have the following advantages:

(i) they show better water solubility than TAXOL® and are thus more appropriate for formulation; and

(ii) they have, in cell culture experiments, been reported to be active also against the proliferation of cells that, due to the activity of the P-glycoprotein efflux pump which renders them multidrug resistant, show resistance to treatment with other chemotherapeutics, e.g. TAXOL® [see Bollag, D. M., et al., “Epothilones, a new class of microtubule-stabilizing agents with a Taxol-like mechanism of action”, Cancer Research 55, 2325-33 (1995); and Bollag D. M., Exp. Opin. Invest. Drugs 6, 867-73 (1997)]; and

(iii) despite apparently sharing the same, or a sterically proximal binding site on the microtubule, the epothilones have been shown to be active against a TAXOL®-resistant ovarian carcinoma cell line with an altered β-tubulin [see Kowalski, R. J., et al., J. Biol. Chem. 272(4), 2534-2541 (1997)].

On the other hand, they are highly toxic and therefore their usefulness in the treatment of cancer in vivo was considered practically impossible [see, for example, PNAS 95, 9642-7 (1998)]. Therefore, the present invention shows in an unexpected way that indeed dosage regimens may be found that allow, on the one hand, to treat tumors with epothilones, especially epothilone B; and on the other hand allow for the treatment of certain patient groups that are unresponsive to other kinds of treatment, be it by multi-drug resistance, as with taxane, e.g. TAXOL®, refractoriness due to multidrug resistance, and/or any other mechanism.

The present invention presents in vivo regimens for a useful treatment with epothilones, preferably epothilone A or especially epothilone B, that allow for the treatment of a tumor disease, e.g. a melanoma, ovarian cancer, pancreas cancer, neuroblastoma, head and neck cancer, bladder cancer, renal, liver, brain, gastric or preferably a colorectal, prostate, breast, lung (especially non-small cell lung) or epidermoid, e.g. epidermoid head and neck, especially mouth, cancer.

While the general treatment schedule allows for the treatment of various tumor types already in front-line treatment, the invention preferably relates to the treatment of tumors that can be expected or have shown to be refractory to treatment with other chemotherapeutics, e.g. standard treatment with one or more other chemotherapeutics, especially with 5-fluorouracil and/or taxane, e.g. TAXOL® treatment.

Surprisingly, it has now been found that even the proliferation of tumor cells and tumors that are refractory to standard treatment with other chemotherapeutics, e.g. 5fluorouracil; andi or to treatment with a member of the taxane class of compounds, most especially TAXOL®, especially of a colorectal tumor, especially one that is also refractory to standard treatment, e.g. with 5-fluorouracil; or of a lung tumor, especially a non-small cell lung cancer; an epidermoid, more preferably epidermoid head and neck, such as mouth, tumor; or a breast tumor; and/or metastasis thereof can be diminished or stopped and that even regression or tumor disappearance is possible.

### DETAILED DESCRIPTION OF THE PREFERRED ASPECTS OF THE INVENTION

The present invention deals preferably with the following subject matter as part of the invention:

Whenever within this whole specification “treatment of a proliferative disease” or of a tumor, cancer or the like is mentioned, there is meant

a) a method of treatment (=for treating) of a proliferative disease, said method comprising the step of administering (for at least one treatment) an epothilone, especially epothilone A and/or B, especially B, (preferably in a pharmaceutically acceptable carrier material) to a warm-blooded animal, especially a human, in need of such treatment, in a dose that allows for the treatment of said disease (=a therapeutically effective amount), preferably in a dose (amount) as specified to be preferred hereinabove and hereinbelow;

b) the use of an epothilone, preferably epothilone A and/or B, especially epothilone B, for the treatment of a proliferative disease, or an epothilone, especially epothilone B, for use in the treatment of said disease (especially in a human);

c) the use of an epothilone, especially epothilone A and/or B, especially epothilone B, for the manufacture of a pharmaceutical preparation for the treatment of a proliferative disease; and/or

d) a pharmaceutical preparation comprising a dose of an epothilone, especially epothilone A and/or B. most especially epothilone B, that is appropriate for the treatment of a proliferative disease; or any combination of a), b), c) and d), in accordance with the subject matter allowable for patenting in a country where this application is filed;

e) a method of using an epothilone for the manufacture of a pharmaceutical preparation for the treatment of a proliferative disease, comprising admixing said epothilone with a pharmaceutically acceptable carrier. In cases where a tumor disease or a specific tumor (e.g. colon tumor, colon carcinoma or colon cancer; or prostate tumor, prostate carcinoma or prostate cancer) are mentioned instead of “proliferative disease”, categories a) to e) are also encompassed, meaning that the respective tumor disease can be filled in under a) to e) above instead of “proliferative disease”, in accordance with the patentable subject matter; preferably, any treatment under a) to e) relates to treatment of humans.

In a first aspect, the present invention relates to an in vivo regimen for the treatment of a proliferative disease, especially a cancer that is refractory to treatment with one or more other chemotherapeutics, especially of the taxane class, like TAXOL®, and/or 5-fluorouracil, where an epothilone, especially epothilone A and/or B, especially epothilone B, is administered daily over about 1 to 14 days, preferably about 1 to 10 days, more preferably over about 1 to 7 days, still more preferably over about 1 to 5 days, even more preferably over about 1, 2, 3, 4 or 5 days, most preferably over about 1 or 5 days, wherein daily administration is by continuous intravenous (i.v.) administration lasting 6 to 24 hours preferably about 8 to 24 hours, more preferably over about 8 to 12 hours, still more preferably over about 16 to 24 hours most preferably about 15 to 17 e.g. 16 or 23 to 24 e.g. 24 hours. For example administration over one day can be a single 24 hour continuous intravenous infusion or administration over 5 days can be five 16 hour continuous intravenous infusions over 5 days, e.g. one 16 hour continuous intravenous infusion on each of the five days.

Preferably administration occurs as herein before described but according to one or more (preferably two to seven) treatment cycle(s), wherein a treatment cycle consists of a dosing period from 1 day to 2 weeks, preferably 1 day to 1 week, most preferably 1 day or 1 week, and a resting period from 6 days to 10 weeks, preferably 1 week to 6 weeks, more preferably 1 week to 4 weeks and most preferably 3 weeks to 4 weeks.

The dosing period is part of a treatment cycle in which an epothilone is administered as herein before described. The administration may occur at the start of the dosing period or at any other time during this period. The dosing period is not the time over which administration occurs it is only the defined period within a treatment cycle that an epothilone is administered. The dosing period may not be shorter than the administration time of the epothilone.

For example, a 3 week treatment cycle could be a 1 week dosing period, in which administration occurs over 1 day wherein administration is by an 8 hour continuous i.v. infusion on day one of this week long dosing period, there is no administration on days 2 to 7 of this week. The 1 week dosing period is followed by a 2 week resting period; or a 3 week treatment cycle could be a 1 week dosing period, in which administration occurs over 5 days wherein administration is by five 8 hour continuous i.v. infusions on days 1 to 5 of this week, no administration occurs on days 6 and 7 of this week. The 1 week dosing period is followed by a 2 week resting period.

In a second aspect, the present invention relates to an in vivo regimen for the treatment of a proliferative disease, especially a cancer that is refractory to treatment with one or more other chemotherapeutics, especially of the taxane class, like TAXOL®, and/or 5-fluorouracil, where an epothilone, especially epothilone A and/or B, especially epothilone B, is administered as herein before described, preferably administered to humans, in a dose of:
dose (mg/m²)=4.0 to 10 (I)

More preferably, the treatment dose is
dose (mg/m²)=5.0 to 10 (II);

even more preferably,
dose (mg/m²)=5.4 to 8 (III);

or most preferably according to the formula IV
dose (mg/m²)=5.4 to 7.0 (IV)

The dose is the total dose administered over 1 to 14 days or the total dose administered per treatment cycle. It is not the dose of each continuous infusion.

Preferably, for about an administration over 1 day the dose is between about 4.0 and about 10, preferably about 5.0 and about 10 mg/m², more preferably about 5.4 and about 8 mg/m²even more preferably about 5.4 and about 7 mg/m² and most preferably about 7 and about 8 mg/m² for about an administration over 5 days the dose is between about 4.0 and about 10, preferably about 5.0 and about 10 mg/m2, more preferably about 5.4 and about 8 mg/m², most preferably about 5.4 and about 6.5 mg/m² even more preferably-about 5.4 and about 6.5 mg/m² and most preferably about 7 and about 8 mg/m².

Preferably, rest periods of more than one week, more preferably of two to ten weeks, more preferably three to six weeks after the preceding treatment may be necessary after for example 3, 4, 6, 8, or more treatment cycles, depending on patient condition, to allow for sufficient recovery from the preceding treatment.

In a third aspect, the invention relates to the in vivo regimen for the treatment of a proliferative disease that is refractory to the treatment with one or more other chemotherapeutics, especially 5-fluorouracil or a microtubule stabilizing agent of the taxane class, especially TAXOL®, for example a multidrug resistant tumor, where an epothilone, especially epothilone B, is administered to a warm-blooded animal, especially a human.

In a fourth aspect, the invention relates to the in vivo regimen for the treatment of a proliferative disease, especially one that is refractory to the treatment with one or more other chemotherapeutics, by combined administration (a) of an epothilone, preferably epothilone A and/or epothilone B, especially epothilone B. in combination with (b) another antitumor chemotherapeutic, preferably the combined treatment being timed so that component (a) and (b) are administered to-a warm-blooded animal, especially a human (especially in need of such treatment), in combination in a quantity which is jointly therapeutically effective against a proliferative disease that preferably can be treated by administration of an epothilone, more preferably epothilone A and/or epothilone B, especially epothilone B; said administration preferably taking place to a human that suffers from a tumor that is refractory to other chemotherapeutic treatment, e.g. treatment especially with 5-fluorouracil or especially with a member of the taxane class of anti-cancer agents, like TAXOL®.

In this regard, the invention also relates to a combination preparation comprising components (a) and (b) as defined in the last paragraph.

The invention also relates to a product which comprises component (a) and component (b) as defined in the second paragraph, starting “In a fourth aspect”, above, in the presence or absence of one or more pharmaceutically acceptable carrier materials, as a combination preparation for simultaneous or chronologically staggered administration to a warm-blooded animal, especially a human, within a period of time which is small enough for the active compounds both of component (a) and of component (b) to mutually enhance antiproliferative activity (especially against proliferating cells) in said warm-blooded animal, for treating a proliferative disease.

The general terms used hereinbefore and hereinafter preferably have the following meanings, if not defined otherwise:

A proliferative disease is mainly a tumor disease (or cancer) (and/or any metastases), wherever the tumor or the metastasis are located), more especially a tumor selected from the group comprising breast cancer, genitourinary cancer, liver, lung cancer, gastrointestinal cancer, epidermoid cancer, melanoma, ovarian cancer, pancreas cancer, neuroblastoma, head and neck cancer (this term, wherever it is used, meaning a head and/or neck cancer, meaning that not only a cancer of head and neck, but also of head or neck is envisaged) or bladder cancer, or in a broader sense renal, brain or gastric cancer; more preferably (i) a tumor selected from a breast tumor; an epidermoid tumor, especially and epidermoid head and neck, preferably mouth, tumor; and a lung tumor, especially a non-small cell lung tumor; or from a gastrointestinal tumor, especially a colorectal tumor; and a genitourinary tumor, especially a prostate tumor (especially a hormone-refractory prostate tumor); or (ii) (more preferably) a proliferative disease that is refractory to the treatment with other chemotherapeutics, especially a corresponding tumor (and/or any metastasis), more especially a tumor selected from the group comprising tumors that are refractory to a standard treatment with (an)other chemotherapeutic(s), especially with 5-fluorouracil and/or (preferably) a microtubule stabilizing agent of the taxane class, most especially TAXOL®, still more preferably a tumor selected from gastrointestinal, e.g. colorectal (especially refractory to standard, e.g. 5-fluorouracil, and/or TAXOL® treatment); and genitourinary, e.g. prostatic tumors and ovarian tumors (and/or a metastasis thereof, especially a metastasis thereof); most preferably a gastrointestinal tumor, especially a colorectal cancer; or (iii) a tumor that is refractory to treatment with other chemotherapeutics due to multidrug resistance, especially refractory to a member of the taxane class of microtubule stabilizing agents, preferably TAXOL®, most especially a multidrug, especially TAXOL®, resistant lung tumor (especially a non-small cell lung tumor), a multidrug resistant breast tumor, or a multidrug resistant epidermoid, preferably epidermoid head and neck, most preferably mouth, tumor.

In a broader sense of the invention, a proliferative disease may furthermore be selected from hyperproliferative conditions such as hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty.

Where hereinbefore and subsequently a tumor, a tumor disease, a carcinoma or a cancer are mentioned, also metastasis in the original organ or tissue and/or in any other location are implied alternatively or in addition, whatever the location of the tumor and/or metastasis is.

The word “refractory” means that the respective proliferative disease (especially a tumor and/or any metastasis thereof), upon treatment with a (meaning at least one) chemotherapeutic other than an epothilone, shows no or only weak antiproliferative response (no or only weak inhibition of tumor growth) after the treatment with such an agent, that is, a tumor that cannot be treated at all or only with unsatisfying results with other (preferably standard) chemotherapeutics (preferably as defined above, especially 5-fluorouracil (especially in the case of colorectal, like colon, cancer), antiandrogens or preferably mitoxantrone (especially in the case of prostate cancer), or antiestrogens, like letrozole (especially in the case of breast cancer); or especially a member of the taxane class of chemotherapeutics, e.g. TAXOTERE® or TAXOL®, in a warm-blooded animal, especially a human; for example the tumor growth is not stopped, only retarded slightly or no regression is found. The present invention, where treatment of refractory tumors and the like is mentioned, is to be understood to encompass not only (a) tumor(s) where one or more chemotherapeutics have already failed during treatment of a patient, but also (a) tumor(s) that can be shown to be refractory by other means, e.g. biopsy and culture in the presence of chemotherapeutics. Where a term like ,,refractory against TAXOL®” is used hereinbefore and hereinafter, this term, in addition to the finished product, is also intended to mean paclitaxel, the active substance of TAXOL®. ,,Refractory to hormone treatment” or ,,hormone refractory”, in the case of a tumor of the genitourinary tract, especially a prostate tumor, means refractory to treatment with an antiandrogen.

TAXOL® preferably stands for the finished product that comprises paclitaxel, but, in a broader sense, is also meant to encompass paclitaxel itself of any other paclitaxel formulation with one or more carrier material(s).

Preferably, the term refractory means that with standard dose a reduction of tumor growth by less than 50% (that is a T/C% value of equal to or more than 50%) is obtained when compared with a control without chemotherapeutic, e.g. by in vivo or in vitro measurements.

Multidrug resistant tumor disease is one where resistance to one or more chemotherapeutics, including those of the taxane class, especially TAXOL®, or the anthracycline class, especially ADRIAMYCIN®, is found. The basis for this resistance is the export via an energy (especially ATP)-dependent pump located on the surface of cells of the respective tumor, especially of the P-glycoprotein family, especially P-glycoprotein (P-gp) itself. In the present invention, alternatively or in addition other mechanisms may cause a tumor to be refractory to treatment with chemotherapeutics other than an epothilone. For example, alterations in the drug target (especially microtubules in the present case), changes in the intracellular metabolism that may inactivate the compound, or changes in the physiology of the cell that would facilitate by-passing or overriding of the mechanism of drug action may lead to such resistance.

By the term “other chemotherapeutic” or ,,standard chemotherapeutic”, there is meant especially any chemotherapeutic other than an epothilone; preferably one as defined in the introduction, especially 5-fluorouracil (especially in the case of colorectal, like colon, cancer), an anti-androgen or mitoxantrone (especially in the case of prostate cancer), or an antiestrogen, like letrozole (especially in the case of breast cancer); especially, the term refers to 5-fluorouracil or (more preferably) to members of the taxane class (especially in the case of ovarian cancer) of microtubule stabilizing agents, such as preferably Taxotere® or more preferably TAXOL®. ,,Standard treatment with other chemotherapeutics”, ,,other chemotherapeutic treatment” or “standard chemotherapy” is referring to treatment with at least one such ,,other” or ,,standard therapeutic”.

By the term epothilone, any epothilone or epothilone derivative is meant. Preferably, the term ,,epothilone” means epothilone A, epothilone B, any epothilone derivative disclosed in WO 98/25929 (which is incorporated by reference), or any mixture thereof; more preferably, it means epothilone A and/or epothilone B, and most preferably it relates to epothilone B.

The administration in all cases mentioned above and below may be made parenterally, especially intravenously, e.g. by infusion or injection. Where subsequently “infusion” is used, this means preferably intravenous or subcutaneous infusion, intravenous is the most preferred mode of administration.

Subsequently, the data for adults are the basis for illustration. However, it goes without saying that the present invention also relates to the treatment of proliferative diseases in pediatrics. The doses must then be corrected in accordance with standard methods and the age, condition and other characteristics of the patient.

The Maximal Tolerated Dose (MTD) is determined according to standard procedures; preferably, in warm-blooded animals the MTD in case of oral or intravenous administration is determined as the Dose of a single bolus administration where no death occurs and a loss of body weight of less than 40, preferably less than 25, percent (%) is found in the treated warm-blooded animal individual (this term here mainly referring to an animal; for humans see below).

The term “single bolus administration” with respect to maximal tolerated dose is a single bolus administration over 5 to 30 minutes.

More preferably, treatment is stopped after the third to eighth, especially after the third to fifth treatment cycle followed by a rest period of one to five weeks before further treatment cycles are resumed.

Administration of component (a), that is epothilones A and/or B, especially B, takes place preferably as described above, especially using one of the special treatment regimens mentioned above.

Administration of component (b) preferably takes place according to treatment schedules that are known to the person skilled in the art.

In one preferred embodiment, component (b) is administered before component (a), preferably in a treatment comprising one or more administrations of component (b) before starting the treatment with component (a), preferably such that treatment with component (b) ends at least two, preferably 5 to 10, e.g. about 5, days prior to treatment with component (a) that is administered one or more times thereafter, preferably one to five, especially one or two times.

In a more preferred embodiment, component (a) is administered on a treatment cycle herein before defined before component (b), preferably In a treatment comprising one administration of component (a) before starting the treatment with component (b), more preferably such that treatment with component (a) ends immediately prior to treatment with component (b) that is administered thereafter.

In a second more preferred embodiment, component (a) is administered according to a treatment cycle herein before defined. Component (b), on the other hand, is administered on a 3 or 4-weekly cycle, with each administration proceeding immediately upon completion of administration of component (a).

By the term “other chemotherapeutic agent” there is meant especially any chemotherapeutic agent that is or can be used in the treatment of tumor diseases, such as chemotherapeutics derived from the following classes:

(A) Alkylating agents, preferably cross-linking chemotherapeutics, preferably bis-alkylating agents,

(B) antitumor antibiotics, preferably doxorubicin (ADRIAMYCIN®, RUBEX®);

(C) antimetabolites;

(D) plant alkaloids;

(E) hormonal agents and antagonists,

(F) biological response modifiers, preferably lymphokines or interferons

(G) inhibitors of protein tyrosine kinases and/or serine/threonine kinases,;

(H) anfisense oligonucleotides or oligonucleotide derivatives; or

(I) miscellaneous agents or agents with other or unknown mechanism of action

(J) monoclonal antibodies.

By the term “jointly therapeutically effective against a proliferative disease that can be treated by administration of epothilone A and/or epothilone B, especially epothilone B”, there is preferably meant a proliferative disease as mentioned above, especially a tumor disease, the response preferably manifesting itself in a diminished proliferation, e.g. diminished tumor growth or even (more preferably) tumor regression or (most preferably) tumor disappearance (“complete response”).

Preferably, the term “quantity which is jointly therapeutically effective against a proliferative disease that can be treated by administration of epothilone A and/or epothilone B, especially epothilone B” means any quantity of the components (a) and (b) of the combinations that, in the combination, is diminishing proliferation of cells responsible for any of the mentioned proliferative diseases, especially tumor (including metastatic) cells (especially diminished tumor growth) or, preferably, even causing regression, more preferably even the partial or complete disappearance, of such cells (especially tumor regression, preferably complete response meaning disappearance of the tumor(s)). This term not only comprises combinations of any component (a) and (b) where (a) and (b) are dosed in such a manner as to be antiproliferatively effective already without combination, but also doses of any such component which alone would show no or only marginal effect but which in combination leads to clearly antiproliferative effects, that is to diminished proliferation or preferably even to regression of the proliferating cells or even to cure from the proliferative disease. In addition, here the term “combination” is not only used to describe fixed combinations of the components, but also any combination of components (a) and (b) for simultaneous or chronologically staggered use within a period of time which is small enough for the active compounds both of component (a) and of component (b) to mutually enhance antiproliferative activity, e.g. in a patient.

By the term “combination preparation comprising component (a) and (b)” there is meant any combination, be it as kit of parts or as a single combined combination, of component (a) and (b) in the form of a pharmaceutical product, that is preferably where a pharmaceutically acceptable carrier material is present. For the preferred carrier materials, see below under “Pharmaceutical Preparations”.

By the term “a product which comprises component (a) and component (b)”, there is preferably meant a product that comprises (a) at least one compound selected from epothilone A and (preferably) epothilone B and (b) at least one other chemotherapeutic agent in the presence or absence of one or more pharmaceutically acceptable carrier materials, as a combination preparation, for simultaneous or chronologically staggered use, preferably within a period of time which is small enough for the active compounds both of component (a) and of component (b) to mutually enhance antiproliferative activity against proliferating cells, especially in a patient, for treating a proliferative disease which responds to such active compounds”, especially a “kit of parts” in the sense that the effective components (a) and (b) of the combination can be dosed independently or by use of different fixed combinations with distinguished amounts of any components (a) and (b) at different time points. The “parts” of the kit of parts can then be administered simultaneously or chronologically staggered, that is at different time points and with equal or different time intervals for any part of the kit of parts, preferably with the condition that the time intervals are chosen such that the effect on the proliferative disease in the combined use of the parts is larger than the effect which would be obtained by use of only any one of component (a) and (b) alone or by use of both in a way that the compounds act independently (e.g. with long enough periods to avoid effects of each of the components on the others), that is, stronger inhibition of proliferation or, preferably, stronger regression or even cure of the proliferative disease is found than when the same dose of only one of components (a) and (b) is administered alone in the same dose or after sufficiently long time intervals that mutual effects of the components (a) and (b) are excluded. That is meant by the term “to mutually enhance antiproliferative activity against proliferating cells, especially in a patient”; preferably, there is meant a mutual enhancement of the effect of the components (a) and (b), especially a synergism and/or the causing of regression of the proliferating cells, up to and including their complete destruction, and especially a strong synergism between components (a) and (b).

By the term “proliferating cells”, especially pathologically or abnormally proliferating cells are meant, such as tumor and/or tumor metastasis cells, especially of tumors as defined herein as being preferred.

Preferred are combinations which show enhanced antiproliferative activity when compared with the single components alone, especially combinations that show synergism (synergistic combinations) or combinations that lead to regression of proliferative tissues and/or cure from proliferative diseases.

The term “synergism” is standing for an effect that is stronger than additive, that is, a stronger effect of the combination of any component (a) with any component (b) than could be reached by the factor of diminution of proliferation obtained from mere multiplication of the factor of diminution of proliferation for any component (a) alone or any component (b) alone when compared to a control without treatment when each (a) and (b) as such, whether alone or in combination, is administered in the same dose as in the single treatment without combination (which does not mean that the dose of (a) must be identical to that of (b), although this may also be the case). As theoretical example for mere illustration, if a component (a) alone gives a growth of tumor cells that is diminished by a factor of 2 in comparison to a control without any treatment and a component (b) alone gives a diminution of growth by a factor of 1.5, then an additive effect would be one where, by combined use of component (a) and component (b), a 3-fold diminution of growth would be found (multiplication of 2 with 1.5). A synergistic effect would for example be present if a more than 3-fold diminution of proliferation is found. The presence of synergism can be shown by this fractional product method [Webb, in: ,,Enzymes and Metabolic Inhibitors”, Vol. 1, 66-73 and 488-512, Academic Press, New York] or alternatively by the isobologram method [see references in: Berenbaum Pharmacol. Rev. 41, 99-141 (1984)], and/or the combination index (Cl) calculation method [Chou et al., Trends Pharmacol. Sci. 4, 450-454 (1983); or Chou et al., New Avenues in Developmental Cancer Chemotherapy; Bristol-Myers Symposium Series, K. R. Harrap and I. A. Connors (eds.), 37-64, New York, Academic Press (1987)].

The term “pharmaceutically acceptable carrier materials” is explained below in the definition of pharmaceutical preparations.

Provided that in the respective molecule salt-forming groups are present, component (b) (other chemotherapeutic(s)) may also be present in the form of salts wherever it is mentioned above or below.

Termination of treatment preferably takes place when either of the following occurs: Disease progression, for example under the RECIST criteria for response; unacceptable toxicity (e.g. to the patient, the investigator, or both); treatment 2 cycles beyond determination of a complete response, for example under the Southwest Oncology Group (SWOG) response criteria; or patient withdrawal of consent.

Salts of components are especially acid addition salts, salts with bases or, when several salt-forming groups are present, optionally also mixed salts or internal salts. Salts are especially the pharmaceutically acceptable, e.g. substantially non-toxic, salts.

Such salts are formed, for example, from chemotherapeutics having an acidic group, for example a carboxy, phosphodiester or phosphorothioate group, and are, for example, their salts with suitable bases, such as non-toxic metal salts derived from metals of groups Ia, Ib, IIa and IIb of the Periodic Table of Elements, especially suitable alkali metal salts, for example lithium, sodium or potassium salts, or ammonium salts, also those salts that are formed with organic amines, such as unsubstituted or hydroxy-substituted mono-, di- or tri-alkylamines, especially mono-, di- or tri-lower alkylamines, or with quaternary ammonium compounds, for example with N-methyl-N-ethylamine, diethylamine, triethylamine, mono-, bis- or tris-(2-hydroxy-lower alkyl)amines, such as mono-, bis- or tris-(2-hydroxyethyl)amine, 2-hydroxy-tert-butylamine or tris(hydroxymethyl)methylamine, N,N-di-lower alkyl-N-(hydroxy-lower alkyl)-amines, such as N,N-dimethyl-N-(2-hydroxyethyl)-amine or tri-(2-hydroxyethyl)-amine, or N-methyl-D-glucamine, or quaternary ammonium salts, such as tetrabutylammonium salts. The chemotherapeutics having a basic group, for example an amino or imino group, can form acid addition salts, for example with inorganic acids, for example a hydrohalic acid, such as hydrochloric acid, sulfuric acid or phosphoric acid, or with organic carboxylic, sulfonic, sulfo or phospho acids or N-substituted sulfamic acids, such as, for example, acetic acid, propionic acid, glycolic acid, succinic acid, maleic acid, hydroxymaleic acid, methylmaleic acid, fumaric acid, malic acid, tartaric acid, gluconic acid, citric acid, or benzoic acid, also with amino acids, for example, a-amino acids, and also with methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, N-cyclohexylsulfamic acid (with formation of the cyclamates) or with other acidic organic compounds, such as ascorbic acid. Compounds having acidic and basic groups can also form internal salts. If more than one salt-forming group is present, it is also possible that mixed salts are present.

Where hereinbefore and hereinafter numerical terms are used, they are meant to include the numbers representing the upper and lower limits. For example, “between 1 and 3” stands for a range “from and including 1 up to and including 3”, and “in the range from 1 to 3” would stand for “from and including 1 up to and including 3”. The same is true where instead of numbers (e.g. 3) words denoting numbers are used (e.g. “three”). Where “comprising” is used, this can preferably be replaced by “consisting essentially of”, more preferably by “consisting of”.

Where “about” is used in connection with a number, this preferably means the number ±15%, more preferably the number plus 5%, most preferably the number itself without “about”. For example, “about 100” would stand for ,,from and including 85 to and including 115”. Where “about” is used in connection with numeric ranges, for example “about 1 to about 3”, or “between about one and about three”, preferably the definition of “about” given for a number in the last sentence is applied to each number defining the start and the end of a range separately. Preferably, where “about” is used in connection with any numerical values, the “about” can be deleted.

“Weekly” stands for “about once a week”, the about here preferably meaning ±1 day (that is, translating into “every 6 to 8 days”); most preferably, “weekly” stands for “once every 7 days”.

In the following preferred embodiments of the invention, the general definitions may be replaced by the more specific definitions given hereinbefore and hereinafter, as appropriate.

(1) The present invention relates especially to the treatment of a proliferative disease, especially a cancer, especially a cancer that is refractory to treatment with other chemotherapeutics and/or a member of the taxane class of anti-cancer agents, especially TAXOL®, more especially one of the preferred diseases as defined above or below, characterized in that an epothilone, especially epothilone A or most especially epothilone B, is administered daily over about 1 to 14 days, preferably about 1 to 10 days, more preferably over about 1 to 7 days, still more preferably over about 1 to 5 days, even more preferably over about 1, 2, 3, 4 or 5 days, most preferably over about 1 or 5 days, wherein daily administration is by continuous intravenous (i.v.) administration lasting 6 to 24 hours preferably about 8 to 24 hours, more preferably over about 8 to 12 hours, still more preferably over about 16 to 24 hours most preferably about 15 to 17 e.g. 16 or 23 to 24 e.g. 24 hours, more than once with a weekly up to six-weekly, preferably three weekly up to four weekly, interval to a human in a dose of:
dose (mg/m²)=4.0 to 10 (I)

More preferably, the treatment dose is
dose (mg/m²)=5.0 to 10 (II);

even more preferably,
dose (mg/m²)=5.4 to 8 (III);

or most preferably according to the formula IV
dose (mg/m²)=5.4 to 7 (IV)

The dose is the total dose administered over 1 to 14 days. It is not the dose of each continuous infusion.

(2) The present invention preferably relates also to the treatment of a tumor disease that is refractory to the treatment with an other chemotherapeutic, especially selected from 5-fluorouracil and preferably a microtubule stabilizing agent of the taxane class, most especially TAXOL®, said tumor being selected from a gastrointestinal, e.g. colorectal; a renal; a genitourinary, e.g. prostatic; a pancreatic; and a brain tumor (and/or any metastasis thereof), most preferably a gastrointestinal tumor, especially a colorectal cancer, more especially a gastrointestinal cancer, especially a colorectal cancer, that is refractory to treatment with a member of the taxane class of anti-cancer agents, especially TAXOL®, or very especially such tumor that is refractory to a standard chemotherapy, such as treatment a standard chemotherapeutic, especially with 5-fluorouracil; or a tumor of the genitourinary tract, especially a prostate cancer, more especially a hormone-refractory prostate cancer, even more especially an ovarian cancer and most especially an ovarian cancer refractory to taxane and/or platinum treatment; where epothilone A and/or B, especially epothilone B, is administered to a warm-blooded animal, especially a human.

(3) The present invention preferably also relates to the treatment of a tumor disease, especially a lung tumor, especially a non-small cell lung carcinoma, especially such lung cancer that is refractory to treatment with a member of the taxane class of anti-cancer agents, especially TAXOL®; a breast tumor, especially one that is multidrug resistant; or an epidermoid tumor, preferably an epidermoid head and neck, especially mouth, tumor, especially if the latter is multidrug resistant and/or resistant to treatment with a member of the taxane class of anti-cancer agents, in particular TAXOL®; where epothilone A and/or B, especially epothilone B, is administered to a warm-blooded animal, especially a human.

(4) The present invention also preferably relates to an in vivo regimen for the treatment of a tumor disease, especially (i) of a tumor of the gastrointestinal tract, most especially a tumor of the colon and/or rectum (colorectal tumor); and/or (ii) a tumor of the genitourinary tract, especially a prostate ovarian tumor (preferably a taxane and/or platinum refractory ovarian tumor); especially where such tumor is refractory to treatment with an other chemotherapeutic, especially 5-fluorouracil and/or one of the taxane class, most especially TAXOL®; where epothilone A and/or B, especially epothilone B, is administered as herein before described once in a dose according to I, II, III or IV, to a human; and, if required, one or more (preferably two to seven) further doses each within the dose range mentioned above are administered in further treatment cycles, preferably each dose after a period of time that allows for sufficient recovery of the treated individual from each preceding dose administration, especially more than one weeks after the preceding treatment, more especially one to 6 weeks, even more especially three to six weeks after the preceding treatment, most especially three to four weeks after that treatment.

More preferably, under (1) to (4) epothilone B is administered over 1 day the dose is between about 4.0 and about 10, preferably about 5.0 and about 10 mg/m², more preferably about 5.4 and about 8 mg/m², even more preferably about 5.4 and about 7 mg/m² and most preferably about 7 and about 8 mg/m² for administration over about 5 days the dose is between about 4.0 and about 10, preferably about 5.0 and about 10 mg/m², more preferably about 5.4 and about 8 mg/m², most preferably about 5.4 and about 6.5 mg/m² even more preferably about 5.4 and about 7 mg/m² and most preferably about 7 and about 8 mg/m². This dose is preferably administered daily, preferably to a human, over about 1 to 14 days, preferably about 1 to 10 days, more preferably over about 1 to 7 days, still more preferably over about 1 to 5 days, even more preferably over about 1, 2, 3, 4 or 5 days, most preferably over about 1 or 5 days, wherein daily administration is by contiuous intravenous (i.v.) administration lasting 6 to 24 hours preferably about 8 to 24 hours, more preferably over about 8 to 12 hours, still more preferably over about 16 to 24 hours most preferably about 15 to 17 e.g. 16 or 23 to 24 e.g. 24 hours

More preferably, said treatment cycle is repeated 1 to 10 cycles, preferably 1 to 7 cycles, until disease progression, unacceptable toxicity, 1 or preferably 2 cycles beyond determination of a complete response, or patient withdrawal of consent for any reason is encountered.

(6) The invention preferably also relates to the in vivo treatment of a tumor disease by combined administration (a) of epothilone A and/or epothilone B. especially epothilone B, in combination with (b) an other chemotherapeutic agent selected from the group consisting of

(A) alkylating agents, preferably cross-linking chemotherapeutics, preferably bis-alkylating agents,

(B) antitumor antibiotics, preferably doxorubicin (ADRIAMYCIN®, RUBEX®);

(C) antimetabolites;

(D) plant alkaloids;

(E) hormonal agents and antagonists,

(F) biological response modifiers, preferably lymphokines or interferons

(G) inhibitors of protein tyrosine kinases and/or serine/threonine kinases,;

(H) antisense oligonucleotides or oligonucleotide derivatives; or

(I) miscellaneous agents or agents with other or unknown mechanism of action; the combined treatment being timed so that component (a) and (b) are combined for simultaneous or chronologically staggered use within a period of time which is small enough for the active compounds both of component (a) and of component (b) to mutually enhance antiproliferative activity, e.g. in a patient.

(J) monoclonal antibodies.

(7) The invention also relates to a product which comprises component (a) and component (b) as defined under (6) above, in the presence or absence of one or more pharmaceutically acceptable carrier materials, as a combination preparation for simultaneous or chronologically staggered administration to a human within a period of time which is small enough for the active compounds both of component (a) and of component (b) to mutually enhance activity against a tumor disease, especially (i) a tumor of the gastrointestinal tract, most especially a tumor of the colon and/or rectum (colorectal tumor); and/or (ii) a tumor of the genitourinary tract, especially an ovarian tumor; especially where such tumor is refractory to treatment with an other chemotherapeutic, especially one of the taxane class, most especially TAXOL®; for treating said tumor disease.

Under (1) to (7) or the subsequent embodiments of the invention, administration of the epothilone, especially epothilone B, preferably takes place by infusion, especially by intravenous infusion.

The following are some especially preferred embodiments of the invention:

A1. The use of an epothilone for the manufacture of a pharmaceutical preparation that is appropriate for administration as herein before described in a dose that is between about 4.0 and 10 mg/m², preferably a dose between 5.0 and 10 mg/m², more preferably a dose between 5.4 to 8 mg/m² or most preferably a dose between 7 and 8 mg/m² in a warm-blooded animal, to said warm blooded animal for the treatment of a proliferative disease especially a proliferative disease that is refractory to the treatment with other chemotherapeutics.

A2. The use according to any one of B1 to B5 where the proliferative disease is a tumor.

A3. The use according to A1 where the proliferative disease is a tumor disease that is refractory to a microtubule stabilizing agent of the taxane class, especially TAXOL®.

A4. The use according to any one of A1 to A3 where the proliferative disease is a colorectal tumor, and/or a metastasis thereof.

A5. The use according to any one of A1 to A3 where the proliferative disease is a ovary tumor, and/or a metastasis thereof; especially a taxane and platinum-refractory tumor.

A6. The use according to A1 to A5 where the epothilone is epothilone A and/or epothilone B, preferably epothilone B.

B1. The use of an epothilone for the manufacture of a pharmaceutical preparation that is appropriate for administration as herein before described and that is appropriate for the combined administration (a) of an epothilone, preferably epothilone A and/or epothilone B, in combination with (b) another antitumor chemotherapeutic to a warm-blooded animal that suffers from a proliferative disease that is refractory to the treatment with other chemotherapeutics, especially a colorectal or prostate tumor and/or a metastasis thereof.

B2. A combination preparation according to B2 comprising (a) epothilone A or preferably epothilone B and (b) one or more other antitumor chemotherapeutics, and a pharmaceutically acceptable carrier.

C1. A product which comprises as component (a) epothilone A and/or B, preferably epothilone B, and as component (b) any other antitumor chemotherapeutic, in the presence or absence of one or more pharmaceutically acceptable carrier materials, as a combination preparation for simultaneous or chronologically staggered administration as herein before described to a warm-blooded animal, especially a human, within a period of time which is small enough for the active compounds both of component (a). and of component (b) to mutually enhance antitumor activity in said warm-blooded animal, for treating a proliferative disease.

The invention relates most especially to the treatment of following tumor/cancer types with epothilone B:

(i) a gastrointestinal, especially a colorectal tumor that is refractory to a representative of the taxane class of anti-cancer agents, in particular TAXOL®; or more especially to the treatment with standard chemotherapy, especially with 5-fluorouracil, and/or TAXOL®.

(ii) a tumor of the genitourinary tract, especially a ovarian tumor, including primary and especially metastatic tumors; more especially if refractory to 5-fluorouracil;

(iii) an epidermoid, more especially epidermoid head and neck, most especially epidermoid mouth tumor, especially one of these that is refractory to treatment with an other chemotherapeutic, especially due to multi-drug resistance, especially to treatment with a member of the taxane class of anti-cancer agents, especially TAXOL®;

(iv) a lung tumor, especially a non-small cell lung cancer, that is refractory to treatment with an other chemotherapeutic, especially due to (mainly) multi-drug resistance, especially to treatment with a member of the taxane class of anti-cancer agents, especially TAXOL®; and/or

(v) a breast tumor, especially a breast tumor that is multidrug resistant, more especially one that is refractory to treatment with a member of the taxane class of anti-cancer agents, especially TAXOL®.

Preferably, the invention relates to the treatment of any one of the above-mentioned tumor types (i) to (v), most preferably to that of (i), (ii), (iv) and (v).

More preferably, the invention relates to the treatment of any of the tumor types mentioned above under (i) to (v), especially to any one of them, by treatment according the a treatment schedual herein before described.

said treatment cycle being repeated one to 8 times, preferably one to 5 times;

where the epothilone B dose is preferably,
dose (mg/m²)=4.0 to 10 (I)

More preferably, the treatment dose is
dose (mg/m²)=5.0 to 10 (II);

even more preferably,
dose (mg/m²)=5.4 to 8 (III);

or most preferably according to the formula IV
dose (mg/m²)=5.4 to 7 (IV)

The dose is the total dose administered over 1 to 14 days. It is not the dose of each continuous infusion. Preferably, for administration over about 1 day the dose is between about 4.0 and about 10, preferably about 5.0 and about 10 mg/m2, more preferably about 5.4 and about 8 mg/m², even more preferably about 5.4 and about 7 mg/m² and most preferably about 7 and about 8 mg/m² for administration over about 5 days the dose is between about 4.0 and about 10, preferably about 5.0 and about 10 mg/m2, more preferably about 5.4 and about 8 mg/m2, most preferably about 5.4 and about 6.5 mg/m2 even more preferably about 5.4 and about 7 mg/m² and most preferably about 7 and about 8 mg/m².

Preferably, rest periods of more than one week, more preferably of two to ten weeks, more preferably three to six weeks after the preceding treatment may be necessary after for example 3, 4, 6, 8, or more treatment cycles, depending on patient condition, to allow for sufficient recovery from the preceding treatment.

Especially preferred are also treatment conditions and formulations in analogy to those mentioned in the Examples.

Pharmaceutical Formulations

The present invention also relates to the use of epothilone A and/or B, especially epothilone A or preferably epothilone B, for the manufacture of a pharmaceutical formulation for use against a tumor disease as defined above; or to a pharmaceutical formulation for the treatment of said tumor disease comprising epothilone A and/or B, especially epothilone A or preferably epothilone B, and a pharmaceutically acceptable carrier.

The invention relates also to pharmaceutical compositions comprising epothilone A and/or epothilone B, especially epothilone B. for the treatment of a proliferative disease, especially a tumor disease defined as being preferred above, and to the preparation of pharmaceutical preparations for said treatment.

Epothilone A and/or B may be used, for example, for the preparation of pharmaceutical compositions that comprise an effective amount of the active ingredient together or in ad-mixture with a significant amount of inorganic or organic, solid or liquid, pharmaceutically acceptable carriers.

The invention relates also to a pharmaceutical composition that is suitable for administration to a warm-blooded animal, especially a human, for the treatment of a proliferative disease as defined hereinbefore, comprising an amount of epothilone A and/or B, especially epothilone B, which is effective for the treatment of said proliferative disease, together with at least one pharmaceutically acceptable carrier.

The pharmaceutical compositions according to the invention are those for parenteral, such as intramuscular or intravenous, administration to a warm-blooded animal (human or animal), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration; preferably, the dose is one of the preferred doses as defined above, being accomodated appropriately where pediatric treatment is intended.

The pharmaceutical compositions comprise from about 0.00002 to about 95%, especially (e.g. in the case of infusion dilutions that are ready for use) of 0.0001 to 0.02%, or (for example in case of infusion concentrates) from about 0.1% to about 95%, preferably from about 20% to about 90%, active ingredient (weight by weight, in each case). Pharmaceutical compositions according to the invention may be, for example, in unit dose form, such as in the form of ampoules, vials.

Preferably, the dose is chosen so as to allow for the treatment regimen based on the dose ranges mentioned above.

The pharmaceutical compositions of the present invention are prepared in a manner known per se, for example by means of conventional dissolving, lyophilizing, mixing, granulating or confectioning processes.

Solutions of the active ingredient, and also suspensions, and especially isotonic aqueous solutions or suspensions, are preferably used, it being possible, for example in the case of lyophilized compositions that comprise the active ingredient alone or together with a pharmaceutically acceptable carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8 to 22, especially from 12 to 22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, β-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375”(polyoxyethylene glycerol trioleate, Gattefossé, Paris), “Miglyol 812”(triglyceride of saturated fatty acids with a chain length of C₈ to C₁₂, Hüls AG, Germany), but especially vegetable oils, such as cottonseed oil, almond oil, olive oil, castor oil, sesame oil, soybean oil and more especially groundnut oil.

The injection or infusion compositions are prepared in customary manner under sterile conditions; the same applies also to introducing the compositions into ampoules or vials and sealing the containers.

Preferred is an infusion formulation comprising epothilone A and/or epothilone B, especially epothilone B, and a pharmaceutically acceptable organic solvent.

The formulation does not require the use of a surfactant. Surfactants such as Cremophor may cause allergic reactions and they also can leach plasticizers from standard PVC containers, tubing and the like. Consequently, when they are employed one is required to use special infusion apparatus, e.g. nitro-glycerine tubing and non-plastizised containers, such as glass, tubing and the like.

The pharmaceutically acceptable organic solvent used in a formulation according to the invention may be chosen from any such organic solvent known in the art. Preferably the solvent is selected from alcohol, e.g. absolute ethanol or ethanol/water mixtures, more preferably 70% ethanol, polyethylene glycol 300, polyethylene glycol 400, polypropylene glycol or N-methylpyrrolidone, most preferably polypropylene glycol or 70% ethanol or especially polyethylene glycol 300.

The Epothilones may preferably be present in the formulation in a concentration of about 0.1 to about 100 mg/ml, more preferably about 1 to about 100 mg/ml, still more preferably about 1 to about 10 mg/ml (especially in infusion concentrates).

Epothilone A and Epothilone B may be used as pure substances or as a mixture of Epothilone A and B. Given the greater anti-tumour activity of Epothilone B it may be employed in a lower concentration than Epothilone A in the formulation. When used in its pure form it is preferable to employ a concentration of Epothilone A of 5 to 100 mg/ml, preferably 10 to 50 mg/ml, whereas when Epothilone B is used in its pure form it is preferably employed in a concentration of 0.1 to 10, more preferably 1 to 10, still more preferably 1 to 2 mg/ml (this number makes reference especially to an infusion concentrate that, before treatment, is diluted accordingly, see below).

Such formulations are conveniently stored in vials or ampoules. Typically the vials or ampoules are made from glass, e.g. borosilicate or soda-lime glass. The vials or ampoules may be of any volume conventional in the art, preferably they are of a size sufficient to accommodate 0.5 to 5 ml of formulation. The formulation is stable for periods of storage of up to 12 to 24 months at temperatures of at least 2 to 8° C.

Formulations must be diluted in an aqueous medium suitable for intravenous administration before the epothilone can be administered to a patient.

The infusion solution preferably must have the same or essentially the same osmotic pressure as body fluid. Accordingly, the aqueous medium preferably contains an isotonic agent which has the effect of rendering the osmotic pressure of the infusion solution the same or essentially the same as body fluid.

The isotonic agent may be selected from any of those known in the art, e.g. mannitol, dextrose, glucose and sodium chloride. Preferably the isotonic agent is glucose or sodium chloride. The isotonic agents may be used in amounts which impart to the infusion solution the same or essentially the same osmotic pressure as body fluid. The precise quantities needed can be determined by routine experimentation and will depend upon the composition of the infusion solution and the nature of the isotonic agent. Selection of a particular isotonic agent is made having regard to the properties of the active agent.

The concentration of isotonic agent in the aqueous medium will depend upon the nature of the particular isotonic agent used. When glucose is used it is preferably used in a concentration of from 1 to 5% w/v, more particularly 5% w/v. When the isotonic agent is sodium chloride it is preferably employed in amounts of up to 1% wlv, in particular 0.9% w/v.

The infusion formulation may be diluted with the aqueous medium. The amount of aqueous medium employed as a diluent is chosen according to the desired concentration of Epothilone in the infusion solution. Preferably the infusion solution is made by mixing a vial or ampoule of infusion concentrate afore-mentioned with an aqueous medium, making the volume up to between 20 ml and 200 ml, preferably between about 50 and about 100 ml, with the aqueous medium.

Infusion solutions may contain other excipients commonly employed in formulations to be administered intravenously. Excipients include antioxidants.

Antioxidants may be employed to protect the epothilone against oxidative degradation. Antioxidants may be chosen from any of those antioxidants known in the art and suitable for intravenous formulations. The amount of antioxidant may be determined by routine experimentation. As an alternative to the addition of an antioxidant, or in addition thereto, the antioxidant effect may be achieved by displacing oxygen (air) from contact with the infusion solution. This may be conveniently carried out by purging the container holding said infusion solution with an inert gas, e.g. nitrogen.

Infusion solutions may be prepared by mixing an ampoule or vial of the formulation with the aqueous medium, e.g. a 5% w/v glucose solution in WFI or especially 0.9% sodium chloride solution in a suitable container, e.g. an infusion bag or bottle.

The infusion solution, once formed, is preferably used immediately or within a short time of being formed, e.g. within 6 hours.

Containers for holding the infusion solutions may be chosen from any conventional container which is non-reactive with the infusion solution. Glass containers made from those glass types afore-mentioned are suitable although it may be preferred to use plastics containers, e.g. plastics infusion bags.

Plastics containers may be principally those composed of thermoplastic polymers. Plastics materials may additionally comprise additives, e.g. plastizisers, fillers, antioxidants, antistatics and other additives conventional in the art.

Plastics suitable for the present invention should be resistant to elevated temperatures required for thermal sterilisation. Preferred plastics infusion bags are those made from PVC plastics materials known in the art.

A wide range of container sizes may be employed. When selecting a container size, consideration may be paid to the solubility of the epothilone in the aqueous medium and the ease of handling and, if appropriate, storage of the container.

It is preferred to use containers which can accommodate between about 250 to 1000 ml of infusion solution, but preferably about 50 to about 120 ml.

Infusion solutions act in a similar fashion to infusion solutions of the microtubule interacting agent paclitaxel, and are beneficial in treating conditions for which paclitaxel might be used. For certain tumors epothilones offer enhanced beneficial effects compared with paclitaxel.

Dosage forms may be conveniently administered intravenously in a dosage of up to 100 mg/m² Epothilone A and up to about 18 mg/m² of Epothilone B. The exact dosage required and the duration of administration will depend upon the seriousness of the condition and the rate of administration, and it is preferably as defined above. As the dose may be delivered intravenously, the dose received and the blood concentration can be determined accurately on the basis of known in vivo and in vitro techniques.

In the case of combinations with an other chemotherapeutic, a fixed combination of two or more components (a) and (b) as defined above or two or more independent formulations (e.g. in a kit of part) are prepared as described above, or the other chemotherapeutic(s) is/are used in standard formulations that are marketed and known to the person of skill in the art.

### EXAMPLES

### Example 1

### A Phase 1, Dose-Finding Study of Single Agent Epothilone B Administered by an 8 Hour Continuous Infusion Over 1 and 5 Days.

Objectives

Primary Objective

To determine the MTD of EP0906 administered in the following regimens and schedules:
   a single bolus i.v. infusion once every 3 weeks and once every 4 weeks
   a continuous infusion for 1 day every 3 weeks and for 1 day every 4 weeks
   a continuous infusion for 5 days every 3 weeks and for 5 days every 4 weeks
      with the use of nutritional supplement and intensive management of CID in patients with locally advanced or metastatic colon cancer.
      Secondary Objectives
To assess the safety and tolerability of EPO906 administered in the above administration regimens and schedules.
To determine preliminary activity of EPO906 administered in the above administration regimens and schedules as defined by overall response rate (CR+PR) according to modified Response Evaluation Criteria In Solid Tumor (RECIST, Post-text supplement 1). To determine early evidence of time to progression (TTP), duration of overall response and time to overall response of EPO906 administered in the above administration regimens and schedules according to the modified RECIST criteria.
To assess the incidence and the severity of diarrhea, to assess recovery time from diarrhea and the variation in body weight over-time in patients receiving EPO906 administered as described above with nutritional supplement and intensive management of CID. To assess functional recovery of the intestinal mucosa using intestinal permeability studies (optional tests in patients in selected centers) described separately in Post text supplement 13. To assess the pathophysiology of diarrhea using various GI tests described in Post-text supplement 3 and 14.
To evaluate the PK of EPO906 in patients receiving a 5-10 min infusion, q3w and q4w at the MTD dose level, and in patients receiving 24 h and 120 h continuous infusion, q3w and q4w at all dose levels.
To investigate tumor-specific mutations and compare gene expression changes in tumor cells with blood cells and plasma for biomarker development.
To perform pharmacogenetic analyses with blood samples obtained from patients treated with EPO906. Pharmacogenetics samples are collected only from those patients who agree to participate in this part of the trial.
   Design

This is an open-label, non-randomized, multicenter, 6 arms, Phase I dose escalation study to evaluate the safety and tolerability of EPO906 in patients with relapsed/refractory advanced colon cancer. To increase tolerability and safety regarding GI-toxicity, the following measures must be employed in all patients and compliance with prophylaxis nutritional supplement and intensive management of CID must be ensured:
Nutritional supplement is composed of several components, each one possessing properties which is useful as dietary management of malnourished colon cancer patients suffering from diarrhea. These will be omega-3 fatty acids, prebiotics, probiotics and antisecretory factor and glutamine.
Intensive management of CID will be based on algorithm for medical management and work-up of EPO906-induced diarrhea and will consist of early detection and diagnosis of diarrhea, appropriate clinical care, use of loperamide, opium tincture, codeine or octreotide.

EPO906 will be administered either as (1) single 5-10 min bolus i.v. infusion once every 3 weeks, or (2) CIV for 1 day (1×24 hours infusion) every 3 weeks, or (3) CIV for 5 days (5×16 hours infusion) every 3 weeks with nutritional supplement plus intensive management of CID as diarrhea control measure. Once MTD is reached in each administration of the q3w regimen, further investigations will be performed to determine MTD in the q4w regimen with EPO906 administered as (4) bolus once every 4 weeks, (5) CIV for 1 day (1×24 hours infusion) once every 4 weeks or (6) CIV for 5 days (5×24 hours infusion) every 4 weeks, with nutritional supplement plus intensive management of CID (Table-1).

Arm 1: Bolus q3w

Arm 2: CIV 1 day q3w

Arm 3: CIV 5 days q3w

Arm 4: Bolus q4w

Arm 5: CIV 1 day q4w

Arm 6: CIV 5 days q4w

TABLE 2
Study design

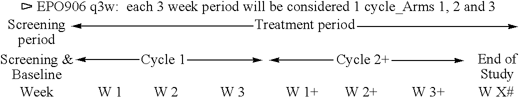

# Patient may come off study at any time. Study completion evaluations should be performed
at this time.
NB: Once the MTD will be reached in the q3w schedule, patients will be recruited into the
respective q4w schedule at the starting dose of MTD defined in the q3w.

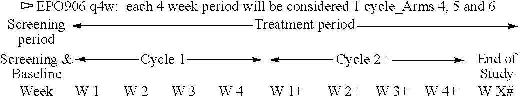

# Patient may come off study at any time. Study completion evaluations should be performed
at this time.

End of Study—Last Patient Last Visit

When all patients entered onto the study have completed at least 6 cycles of study drug, the last patient last visit will be declared, database will close and the Clinical Study Report will be written.

For patients who are CR, PR or SD, and in the investigators' opinion, would continue to benefit from study drug, patients will continue to receive additional treatment with EPO906 as a single agent and followed according to standard of care. If the patient starts a new antineoplastic therapy, the patient is to be discontinued.

Adverse events, dosing administration and tumor response data will continue to be collected after data base closure. An addendum to the Clinical Study Report will be completed after all patients have discontinued.

Discussion of Design

This will be a phase I study using the “3+3 ”design (Storer 1989) to determine MTD of EPO906 administered.

The purpose of this study is to find an optimal administration regimen of EPO906 with the least toxicity and highest MTD to improve the safety and efficacy profile. This will be achieved with measures such as intensive management of CID (adapted from JCO 1998 guidelines for treatment of CID, Wadler 1998). Further supportive measures for dietary management of malnourished colon cancer patients suffering from diarrhea will be prophylactic administration of nutritional supplement.

For the determination of MTD, the DLTs occurring in cycles 1 and 2 are considered to better evaluate the dose and dosing interval than relying only on cycle 1 observations.

The number of patients needed to define the MTD will depend on how the dose escalation proceeds. Approximately 60 patients for the q3w dose escalation in the first 3 arms and approximately 30 patients for the q4w administration in the last 3 arms. Moreover, at the highest MTD reached in the bolus and CIV arms, 6 more patients will be needed, evaluable for PK purposes.

If at the lowest dose in the bolus arm, ⅔ or 2/6 patients show DLT at the first cycle, then this arm is stopped. If at the lowest dose in the bolus arm, ⅔ or 2/6 patients show DLT at the second cycle, then the lowest dose is tested in the bolus arm q4w.

The CIV administration arms may start at a later time than the bolus arm, being dependent on the availability of stability data for EPO906 at low concentrations.

Dose de-escalation below 6.5 mg/m2 will be attempted only for the CIV arms of the study (if until then, stability data for these very low concentrations is available). For the bolus administration no de-escalation will be attempted because there is sufficient data on lower doses available.

Patient Population

Patients with advanced colon cancer (ACC) without ileostomy or colostomy or history of abdominal or pelvic irradiation who relapsed or are refractory after 5-FU or 5-FU in combination with established cytotoxic compounds (irinotecan or oxaliplatin or other cytotoxic compounds used in colon cancer). Patients may have had previous adjuvant or neoadjuvant treatment with 5-FU or 5-FU in combination with established cytotoxic drugs or previous treatment with cetuximab (anti EGFR) or regionally administered chemotherapy.

This study needs approximately 90 evaluable patients to find the MTD depending on the toxicities seen.

Inclusion and exclusion criteria

Inclusion criteria

The following criteria are to be met for inclusion into the study:
Patients with histologically or cytologically confirmed diagnosis of locally advanced progressive or metastatic colon cancer.
Patients should have at least one measurable lesion as defined by modified RECIST criteria (Post-text supplement 1).
Patients who had first-line treatment for metastatic disease with 5-FU or 5-FU in combination with established cytotoxic drugs.
Male or female patients of any ethnic group≧18 years old.
Patients with Performance Status<2 (WHO scale).
Patients with life expectancy of at least 3 months.
Patients with no impairment of hepatic, renal or hematological function as defined by the following parameters:
   Hb≧9.0 g/dL
   platelet count≧100×109/L (untransfused)
   ANC≧1.5×109/L
   serum ALT (SGPT) or AST (SGOT)≦2.5×ULN (≦5×ULN if liver metastases are present)
   serum total bilirubin≦1.5×ULN >serum creafinine≦2.0×ULN
Female patients must have a negative serum pregnancy test (not applicable to patients with bilateral oophorectomy and/or hysterectomy or those patients who are postmenopausal).
All adults of reproductive potential must agree to use an effective method of birth control during the study and for at least 3 months following termination of treatment.
All patients must use a barrier method for contraception for sexual intercourse or avoid this for the first 5 days after EPO906 infusion.
Written informed consent must be obtained.
   Exclusion Criteria
Patients who have not fully recovered from surgery for any cause.
Patients who have received any chemotherapy, immunotherapy or any investigational agent (except cetuximab) within 28 days prior to study entry.
Patients who have received previous pelvic or abdominal radiotherapy.
Patients with ileo- or colostomy.
Patients with a concurrent malignancy, unless they have remained free of the disease attributed to the malignancy for >3 years. Patients with a history of non-melanomatous skin cancer and cervical carcinoma in situ are excluded only if there is evidence of active disease.
Patients receiving hematopoletic growth factors except erythropoietin (refer section 3.4.4).
Patients taking Coumadin® or other agents containing warfarin in doses higher than 1 mg per day. Low dose of Coumadin®) (1 mg or less) administered prophylactically for maintenance of indwelling lines or ports is allowed.
Patients with the presence of active or suspected acute or chronic uncontrolled infection, including abscess or fistulae.
Presence of another nonmalignant disease which in the opinion of the investigator is incompatible with the protocol.
Patients with clinical signs of symptomatic brain metastases or leptomeningeal disease.
Patients with cardiac disease, with an abnormal ECG at baseline, classified under the New York Heart Association classification of III or IV.
Patient with a known diagnosis of human immunodeficiency virus (HIV) infection.
Patients with a history of noncompliance to medical regimens or patients who are considered potentially unreliable.
Pregnant or lactating females.
Patients with diarrhea>grade 1.
Patients who are on prophylactic loperamide treatment.
Patients with peripheral polyneuropathy>grade 1.
   Treatments

EPO906 will be administered either as a single 5-10 min bolus i.v. infusion once every 3 weeks and once every 4 weeks, or administered as a continuous infusion for 1 day (1×24 hours) every 3 weeks and every 4 weeks, or for 5 days (5×24 hours) every 3 weeks and every 4 weeks.

EPO906 will be administered, for each schedule, at a starting total dose per cycle of 6.5 mg/m2 with body surface area calculation based on actual body weight. Dose adjustments should be made for any change in body weight>10% occurring throughout the study. A nomogram for the assessment of body surface area from the patient's height and weight is provided in Post-text supplement 2 of the protocol.

Safety

Safety population includes all patients who participated in the study who have received at least one dose of study medication (EPO906) and have had at least one safety evaluation after administration of study medication. All safety evaluations and analyses will be performed on this patient population.

Efficacy

This population includes all patients who received treatment at least once and who are not severe protocol violators (e.g. patients who were included wrongly).

Efficacy is not the primary objective of this dose finding study. However, the best overall response (CR, PR, SD, PD or UNK) for each patient will be summarized. The overall response rate will be defined as the proportion of patients whose best overall response was complete response (CR) or partial response (PR). Confidence intervals for the overall response rate (CR+PR) will be presented. All efficacy parameters will be presented by administration arm.

Patients who discontinue for a disease or treatment-related reason (e.g. death, adverse event, clinical disease progression, etc.) prior to the first assessment of overall tumor response are classified as non-responders in the analysis of tumor response.

The following pharmacokinetic parameters characterizing the disposition of EPO906 will be calculated from each blood concentration-time profile:
AUC0—Area under the concentration-time curve from time zero to the last
tz sampling time (tz) at which the concentration was above LOQ
AUC0—Area under the concentration-time curve from time zero to infinity
∝ Note: it may not be feasible to calculate this value.
Cmax Maximum (peak) blood drug concentration immediately after the end of infusion for 5-10 min infusion and immediately before the end of infusion for 24 h and 120 h continuous infusion
CL Total body clearance of EP0906 from blood Note: it may not be feasible to calculate this value.
t½ Elimination half-life associated with the terminal slope (λz) of a semilogarithmic concentration-time curve Note: it may not be feasible to calculate this value.
R Drug accumulation by AUC_{∫} dose 4/AUC_{∫} dose 1 for 5-10 min infusion schedule
Vss Apparent volume of distribution at steady state for EPO906

Pharmacokinetic and Pharmacodynamic Analysis

Exploratory evaluation of relationship between pharmacokinetic measurements and clinical outcome (response, toxicity, biomarkers, etc) will be performed if feasible.

Statistical Methods

A 3+3 design for dose escalation will be used (Storer 1989). For the purpose of determining the MTD, six dose levels of EPO906 will be under consideration. These are 6.5 mg/m2, 7.0 mg/m2, 7.5 mg/m2, and 8.0 mg/m2, 8.5 mg/m2 and 9.0 mg/m2. Dose escalation will be based on toxicities from the first and second cycle for each cohort of patients. Intra-patient dose escalation will not be permitted.

The MTD will be defined for each of the 6 arms, starting with the q3w schedule.

MTD is defined as the dose level immediately below that at which DLT is observed in at least two out of three to six patients in each of the 3 arms with the q3w schedule. When the MTD is found for the q3w schedule, the q4w schedule arms are opened starting with the MTD dose which was found in the respective q3w arm.

The number of patients needed to define the MTD will depend on how the dose escalation proceeds.

1. A method for treating a proliferative disease, said method comprising the step of daily administration over 1 to 14 days, of a therapeutically effective amount of an epothilone, together with a pharmaceutically acceptable carrier, to a warm-blooded animal in need of such treatment, wherein the daily is by continuous intravenous (i.v.) administration lasting 6 to 24 hours.

2. The method according to claim 1, where the epothilone is epothilone B.

3. The method according to claim 1 where an epothilone is used in more than one treatment cycle, wherein a treatment cycle consists of a dosing period from 1 day to 2 weeks and a resting period from 6 days to 10 weeks, wherein the dosing period may not be shorter than the administration over 1 to 14 days.

4. The method according to claim 1 where epothilone B is used in a dose in humans that is between 4.0 mg/m² and 10 mg/m².

5. The method according to claim 1 where epothilone B is used in a dose in humans that is between 5 mg/m² and 10 mg/m².

6. The method according to claim 1 where epothilone B is used in a dose that is between between 5.4 mg/m² and 8 mg/m².

7. The method according to claim 1 to where epothilone B is used in a dose that is between between 7 mg/m² and 8 mg/m².

8. The method for according to claim 1 where epothilone B is administered by intravenous infusion over 1 to 10 days.

9. The method according to claim 1 where the epothilone is administered by intravenous infusion over 1 to 7 days.

10. The method according to claim 1 where the epothilone is administered by intravenous infusion over 1 to 5 days.

11. The method according to claim 1 where the epothilone is administered by intravenous infusion over 1 day.

12. The method according to claim 1 where the epothilone is administered by intravenous infusion over 5 days.

13. The method according to claim 1 wherein the proliferative disease is refractory to treatment with one or more chemotherapeutics other than an epothilone, where an epothilone, especially epothilone B, is administered to a human in need of such treatment in a dose that is appropriate for the treatment of said disease.

14. The method according to claim 13 where the refractory tumor to be treated is selected from the group consisting of lung, colorectal, prostate, ovarian, breast or epidermoid head or neck tumors.

15. The method according to claim 13 wherein the tumor to be treated is a colorectal tumor that is refractory to 5-fluorouracil.

16. The method according to claim 15 wherein the colorectal tumor to be treated is in addition refractory to at least one other standard chemotherapeutic.

17. The method according to claim 16 where the tumor to be treated is a colorectal tumor that is refractory to TAXOL and 5-fluorouracil treatment.

18. The method according to claim 13 where the tumor to be treated is an ovarian tumor, and/or any metastasis thereof, refractory to 5-fluorouracil.

19. The method according to claim 13 where the tumor to be treated is an epidermoid head or neck tumor that is refractory to treatment with at least one other chemotherapeutic.

20. The method according to claim 19 where the epidermoid head or neck tumor is refractory to treatment with TAXOL.

21. The method according to claim 13, where the tumor to be treated is a lung tumor that is refractory to treatment with at least one other chemotherapeutic.

22. The method according to claim 21 where the tumor to be treated is a non-small cell lung cancer.

23. The method according to claim 22 where the non-small cell lung cancer is refractory to treatment with TAXOL.

24. The method according to claim 13 where the tumor to be treated is a breast tumor.

25. The method according to claim 13 wherein the tumor to be treated is a colorectal tumor that is refractory to standard chemotherapy.

26. The method according to claim 13 where the tumor to be treated is an epidermoid head or neck tumor refractory to treatment with at least one other chemotherapeutic due to multi-drug resistance.

27. The method according to claim 1 where the proliferative disease to be treated is selected from the group consisting of a colorectal tumor, a tumor of the genitourinary tract, an epidermoid tumor, a lung tumor and a breast tumor.

28. The method according to claim 27 where the proliferative disease to be treated is a colorectal tumor that is refractory to at least 5-fluorouracil and/or to standard chemotherapy.

29. The method according to claim 27 where the proliferative disease to be treated is an ovarian tumor.

30. The method according to claim 29 where the ovarian tumor is refractory to 5-fluorouracil.

31. The method according to claim 27 where the proliferative disease is an epidermoid head or neck tumor.

32. The method according to claim 31 where the head or neck tumor is multidrug-resistant.

33. The method according to claim 27 where the proliferative disease is a non-small cell lung tumor.

34. The method according to claim 33 where the non-small cell lung tumor is refractory to treatment with a member of the taxane class of anti-cancer agents.

35. The method according to claim 27 where the proliferative disease is a breast tumor.

36. The method according to claim 35 where the breast tumor is refractory to treatment with at least one member of the taxane class of anti-cancer agents.

37. The method according to any one of claim 1 where the proliferative disease to be treated is a multidrug resistant tumor.

38. The method according to claim 1 where the proliferative disease to be treated is selected from the group consisting of a melanoma, ovarian cancer, pancreas cancer, neuroblastoma, head or neck cancer, bladder cancer, renal cancer, brain cancer and gastric cancer.

39. The method according to claim 1, further comprising the step of administering (a) epothilone B in combination with (b) another antitumor therapeutic, the combined treatment being so timed that component (a) and component (b) are administered to a human in need of such treatment in combination and in a quantity that is jointly therapeutically effective against said proliferative disease.

40. The method according to claim 1, where the proliferative disease is a tumor that is refractory to the treatment with an anti-cancer agent of the taxane class, said tumor being selected from the group consisting of a colorectal, an ovarian, a pancreatic and a brain tumor.

41. The method according to claim 1 where the proliferative disease is a multidrug resistant non-small cell lung carcinoma, a multidrug resistant breast tumor, or a multidrug resistant epidermoid head and neck tumor.
